(19) United States
(12) Patent Application Publication
(10) Pub. No.: US 2023/0219869 A1
(43) Pub. Date: Jul. 13, 2023

(54) PROCESSES FOR FRACTIONATING A GASEOUS MATERIAL WITH A FACILITATED TRANSPORT MEMBRANE

(71) Applicant: IMTEX MEMBRANES CORP, Mississauga (CA)

(72) Inventors: Xianshe FENG, Mississauga (CA); Karlis VASARAIS, Mississauga (CA); Jamie HUGHES, Mississauga (CA)

(21) Appl. No.: 18/148,870

(22) Filed: Dec. 30, 2022

Related U.S. Application Data
(63) Continuation of application No. PCT/CA21/50909, filed on Jul. 2, 2021.
(60) Provisional application No. 63/047,903, filed on Jul. 2, 2020.

(51) Int. Cl. C07C 7/144; B01D 53/22

(52) U.S. Cl. CPC C07C 7/144; B01D 53/228

(57) ABSTRACT

There is provided a process for producing a target material-enriched product from a target material-comprising gaseous feed material, wherein the target material-comprising gaseous feed material includes a carrier agent-interacting material, comprising: treating the target material-comprising gaseous feed material for effecting depletion of the carrier agent-interacting material within the target material-comprising gaseous feed material, with effect that a carrier agent-interacting material-depleted gaseous material is produced; and fractionating the carrier agent-interacting material-depleted gaseous material via a membrane, with effect that a product is obtained that is enriched in the target material relative to the target material-comprising gaseous feed material. The membrane includes a carrier agent to which the carrier agent-interacting agent is detrimental in response to emplacement of the carrier agent-interacting agent in mass transfer communication with the carrier agent.

### CROSS-REFERENCE TO RELATED APPLICATION

This application is a continuation of International Patent Application No. PCT/CA2021/050909 filed Jul. 2, 2021, titled PROCESSES FOR FRACTIONATING A GASEOUS MATERIAL WITH A FACILITATED TRANSPORT MEMBRANE, which claims the benefits of priority to U.S. Provisional Patent Application No. 63/047,903, filed Jul. 2, 2020, titled PROCESSES FOR FRACTIONATING A GASEOUS MATERIAL WITH A FACILITATED TRANSPORT MEMBRANE. The contents of International Patent Application No. PCT/CA2021/050909, and U.S. Provisional Patent Application No. 63/047,903 are hereby expressly incorporated into the present application by reference in their entirety.

### FIELD

This relates to improving the performance of permeation processes.

### BACKGROUND

Membrane-based separation has proved to be an efficient technology for gaseous separations. Some of the mechanisms for facilitating selective permeation of material through the membrane involve bonding with a carrier that is embodied within the membrane. This carrier forms a reversible complex with materials within a gaseous material, enabling preferential transport of such materials across the membrane, thereby enabling fractionation of the gaseous material. Some materials, if present in gaseous materials, could undesirably react with the carrier material, and thereby degrade the performance of the membrane.

### SUMMARY

In one aspect, there is provided a process for producing a target material-enriched product from a target material-comprising gaseous feed material, wherein the target material-comprising gaseous feed material includes a carrier agent-interacting material, comprising: treating the target material-comprising gaseous feed material for effecting depletion of the carrier agent-interacting material within the target material-comprising gaseous feed material, with effect that a carrier agent-interacting material-depleted gaseous material is produced; and fractionating the carrier agent-interacting material-depleted gaseous material via a membrane, with effect that a product is obtained that is enriched in the target material relative to the target material-comprising gaseous feed material. The membrane includes a carrier agent to which the carrier agent-interacting agent is detrimental in response to emplacement of the carrier agent-interacting agent in mass transfer communication with the carrier agent.

In another aspect, there is provided a process for fractionating a gaseous feed material including an olefin-comprising material, and the olefin-comprising material includes a first olefin and a second olefin. The fractionating of the gaseous feed material is effected via a membrane based on relative permeability as between the first and second olefins. The first olefin has a total number of “X” carbon atoms and the second olefin as a total number of “Y” carbon atoms. Each one of “X” and “Y”, independently, is a whole number that is equal to, or greater than, two (2). “X” is greater than “Y”. The first olefin is characterized by a first permeability coefficient, and the second olefin is characterized by a second permeability coefficient. The first permeability coefficient is greater than the second permeability coefficient.

In another aspect, there is provided a process for recovering olefin-comprising material from a methanol-comprising material, comprising: converting the methanol-comprising material to a gaseous material via a methanol-to-olefin (“MTO”) process, wherein the gaseous material includes olefin-comprising material; fractionating the gaseous material, via a membrane, with effect that a permeate is produced, and the permeate is defined by an olefin material-enriched product, that is enriched in olefin-comprising material relative to the gaseous material, and a retentate is produced, and the retentate is defined by an olefin material-depleted product that is depleted in olefin-comprising material relative to the gaseous material; and recycling at least a portion of the permeate to the MTO process.

In another aspect, there is provided a process for recovering olefin-comprising material from a methanol-comprising material, comprising: converting the methanol-comprising material to a gaseous material via a MTO process, wherein the gaseous material includes olefin-comprising material; fractionating the gaseous material, via a membrane, with effect that a permeate is produced, and the permeate is defined by an olefin material-enriched product, that is enriched in olefin-comprising material relative to the gaseous material, and a retentate is produced, and the retentate is defined by an olefin material-depleted product that is depleted in olefin-comprising material relative to the gaseous material; and recycling at least a portion of the retentate to the MTO process.

### BRIEF DESCRIPTION OF DRAWINGS

The preferred embodiments will now be described with the following accompanying drawings:

FIG. 1 is a schematic illustration of an embodiment of a system in which is practised an embodiment of the process of the present disclosure;

FIG. 2 is a schematic illustration of another embodiment of a system in which is practised an embodiment of the process of the present disclosure;

FIG. 3 is a schematic illustration of another embodiment of a system in which is practised an embodiment of the process of the present disclosure;

FIG. 4 is a schematic illustration of another embodiment of a system in which is practised an embodiment of the process of the present disclosure;

FIG. 5 is a schematic illustration of another embodiment of a system in which is practised an embodiment of the process of the present disclosure;

FIG. 6 is a schematic illustration of another embodiment of a system in which is practised an embodiment of the process of the present disclosure;

FIG. 7 is a schematic illustration of another embodiment of a system in which is practised an embodiment of the process of the present disclosure;

FIG. 8 is a schematic illustration of another embodiment of a system in which is practised an embodiment of the process of the present disclosure; and

FIG. 9 is a schematic illustration of another embodiment of a system in which is practised an embodiment of the process of the present disclosure.

### DETAILED DESCRIPTION

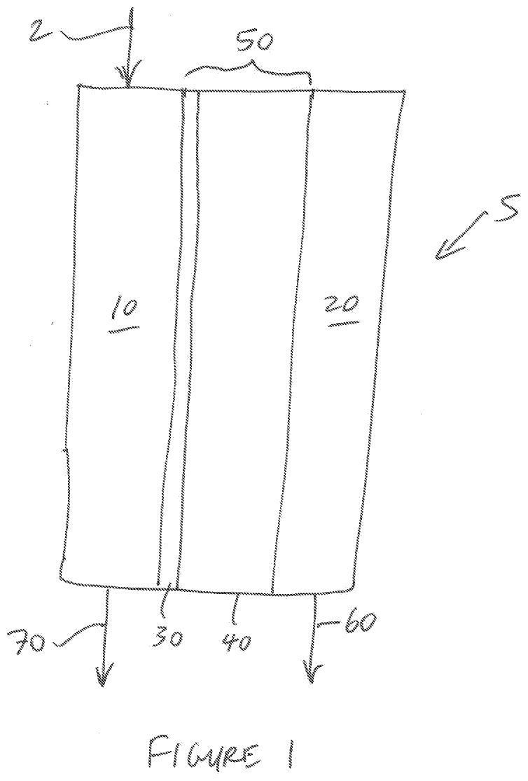

Referring to FIG. 1, there is provided a process for fractionating a gaseous feed material 2 via a membrane 30. In some embodiments, for example, the process is implemented within a system 5 including a feed material receiving space 10, a membrane 30, and a permeate-receiving space 20. The gaseous feed material 2 is supplied to the feed material receiving space 10 that is disposed in mass transfer communication with the permeate receiving space 20 through the membrane 30.

The gaseous material includes a first material, characterized by a first permeability coefficient, and a second material, characterized by a second permeability coefficient, wherein the first permeability coefficient is greater than the second permeability coefficient. The fractionation of the gaseous feed material is with effect that the first material becomes enriched within the permeate 60, and the second material becomes enriched within the retentate 70. In this respect, the fractionation is based on the relative permeabilities, of the constituent materials of the gaseous feed material, through the membrane 30. In some embodiments, for example, the fractionation, is with effect that the separation factor (as measured by the ratio of the permeability coefficient of the first material to the permeability coefficient of the second material) for the separation of the first material from the second material, based on the first material is at least two (2).

The membrane 30 is a facilitated transport membrane and includes a carrier agent. The carrier agent is provided for facilitating transport of material through the membrane. In some embodiments, for example, the carrier agent is uniformly distributed throughout the membrane. In some embodiments, for example, the facilitated transport membrane 30 includes between 15 weight percent of carrier agent, based on the total weight of the membrane, and 70 weight percent of carrier agent, based on the total weight of the membrane (on a wet basis). In some embodiments, for example, the facilitated transport membrane 30 includes between 40 weight percent of carrier agent, based on the total weight of the membrane, and 65 weight percent of carrier agent, based on the total weight of the membrane (on a wet basis). In some embodiments, for example, the facilitated transport membrane 30 includes between 45 weight percent of carrier agent, based on the total weight of the membrane, and 60 weight percent of carrier agent, based on the total weight of the membrane (on a wet basis).

In some embodiments, for example, the carrier agent includes at least one metal cation. In some embodiments, for example, the carrier agent includes silver ion. In some embodiments, for example, the carrier agent includes cuprous ion. In some embodiments, for example, the carrier agent includes silver ion and, in some of these embodiments, for example, the liquid material includes dissolved silver nitrate, and the carrier agent includes the silver ion of the silver nitrate.

In some embodiments, for example, the facilitated transport membrane 30 includes a support phase and the carrier agent is associated with the support phase, and, for at least a portion of the carrier agent, the association is with effect that the carrier agent is covalently bonded to the support phase.

In some embodiments, for example, the facilitated transport membrane 30 includes a support phase, and the carrier agent is associated with the support phase, and, for at least a portion of the carrier agent, the association is with effect that the carrier agent and the support phase define a complex, such as, for example, a chelation complex.

In some embodiments, for example, the facilitated transport membrane 30 includes a support phase and the carrier agent is associated with the support phase, and, for a portion of the carrier agent, the association is with effect that the carrier agent is covalently bonded to the support phase, and, for another portion of the carrier agent, the association is with effect that the carrier agent and the support phase define a complex, such as, for example, a chelation complex,

In some embodiments, for example, the support phase, of the membrane 30 includes polymeric material, and the polymeric material includes at least one polymer compound. In some embodiments, for example, each one of the at least one polymer compound, independently, is hydrophilic.

In those embodiments where the facilitated transport membrane 30 includes a support phase and the carrier agent is associated with the support phase, and the association includes one of: (i) an association that is with effect that at least a portion of the carrier agent is covalently bonded to the support phase, (ii) an association that is with effect that at least a portion of the carrier agent and the support phase define a complex, such as, for example, a chelation complex, and (iii) an association between a portion of the carrier agent and the support phase that is with effect that at least a portion of the carrier agent is covalently bonded to the support phase, and an association between at least another portion of the carrier agent and the support phase that is with effect that the carrier agent and the support phase define a complex, such as, for example, a chelation complex

In some embodiments, for example, each one of the at least one polymer compound, independently, has a number average molecular weight of between 20,000 and 1,000,000. In some embodiments, for example, the polymeric material includes polysaccharide material. In this respect, in some embodiments, for example, the polysaccharide material includes one or more polysaccharides. Suitable polysaccharides include natural polysaccharides such as alginic acid, pectic acid, chondroitin, hyaluronic acid and xanthan gum; cellulose, chitin, pullulan, derivatives of natural polysachharides such as C1-6 esters, esters, ether and alkylcarboxy derivatives thereof, and phosphates of these natural polysaccharide such as partially methylesterified alginic acid, carbomethoxylated alginic acid, phosphorylated alginic acid and aminated alginic acid, salts of anionic cellulose derivatives such as carboxymethyl cellulose, cellulose sulfate, cellulose phosphate, sulfoethyl cellulose and phosphonoethyl cellulose, and semi-synthetic polysaccharides such as guar gum phosphate and chitin phosphate. Specific examples of membranes of polysaccharides include those composed of salts of chitosan and its derivatives (including salts of chitosan) such as N-acetylated chitosan, chitosan phosphate and carbomethoxylated chitosan. Of these, membranes composed of alginic acid, and salts and derivatives thereof, chitosan and salts and derivatives thereof cellulose and derivatives thereof are preferred in view of their film-formability, mechanical strength and film functions, as well as gel formation and swellability (the tendency to be swollen when exposed to water).

In some embodiments, for example, the facilitated transport membrane 30 further includes a liquid phase, and the carrier agent is dissolved within the liquid phase. In some embodiments, for example, the liquid phase is aqueous, such that the liquid phase includes an aqueous solution and the carrier agent is dissolved within the aqueous solution.

In some embodiments, for example, the liquid phase is associated with the support phase. Association includes any type of interaction that is effective for contributing to the facilitation of the fractionation of the gaseous feed material by the membrane 30, including one or more of: (i) chemical bonds (for example, covalent, ionic and hydrogen bonds), (ii) Van der Waals forces, (iii) polar and non-polar interactions through other physical constraints provided by molecular structure, and (iv) interactions through physical mixing. In those embodiments where the support phase is a microporous film, and the microporous film includes polymeric material, in some of these embodiments, for example, the association is with effect that a gel is defined. In some embodiments, for example, the gel includes a hydrogel. In some embodiments, for example, the association is with effect that the polymeric phase is swollen.

In some embodiments, for example, the liquid phase is defined by a continuous liquid phase domain, and the continuous liquid phase domain is encapsulated within the polymeric material of the support phase.

In those embodiments where the facilitated transport membrane 30 includes a hydrogel, in some of these embodiments, for example, the hydrogel includes one or more polysaccharides, and also includes one or more other polymeric compounds. In this respect, in some embodiments, for example, the membranes is comprised of blends of a major amount (e.g. at least 60 weight %, based on the total weight of the membrane) of one or more polysaccharides and lesser amounts (e.g. up to 40 weight %, based on the total weight of the membrane) of one or more other compatible polymeric compounds, such as, for example, polyvinyl alcohol (PVA), or neutral polysaccharides such as starch and pullulan. In some embodiments, for example, the membrane is comprised of grafted ionized polysaccharides obtained by grafting a hydrophilic vinyl monomer such as acrylic acid.

In some embodiments, for example, the facilitated transport membrane 30 is a supported liquid membrane. In this respect, the supported liquid membrane includes a liquid phase that is associated with a support phase, and the association includes disposition of the liquid phase within pores of the support phase with effect that capillary forces are established for interfering with relative displacement between the liquid phase and the support phase. In some embodiments, the support phase includes hydrophilic material. In some embodiments, for example, the support phase is a microporous film. In some embodiments, for example, the pore size of the microporous film which yields a capillary pressure that is greater than the pressure differential applied to the membrane 30 during the process. In some embodiments, for example, the microporous film includes polymeric material. Suitable polymeric materials include polysulphone, sulphonated polysulphone, polyamide, sulphonated polyamide, cellulose acetate, cellulose triacetate, and polyacrylonitrile. In some embodiments, for example, the microporous film includes inorganic material.

In some embodiments, for example, the membrane 30 is supported on a substrate 40 such that a composite membrane 50 is obtained. In some embodiments, for example, the membrane and the substrate co-operate with effect that the membrane is adsorbed to the substrate. In some embodiments, for example, the adsorption includes adhesion. Suitable substrates include films and non-woven supports. Suitable substrates also include ultrafiltration membranes and nanofiltration membranes, with pore size of between 1 and 500 nanometres, such as, for example, between 5 and 300 nanometres.

Suitable substrate materials include polyesters, polysulphones, polyethersulphones, polyimides, polyamides, polycarbonates, polyacrylonitriles, cellulose acetate, and any combination thereof. Substrate material can also be fine pore ceramic, glass and/or metal. In some embodiments, for example, the above-described materials may be pre-coated with a non-selective thin layer of another polymeric material (e.g. polysulphone sparingly coated with a more hydrophilic macromolecule such as polyvinyl alcohol, polyethylene glycol, or polyvinylpyrrolidone) such that the obtained substrate includes two layers. In some embodiments, for example, the coating is for protecting the pores of the underlying layer. In some embodiments, for example, the coating is for facilitating the subsequent application and adsorption of the membrane 30 to the substrate 40.

The composite membrane 50 can be embodied in any one of several configurations, including flat sheet (assembled in a plate and frame or spiral wound module), tubular, or hollow fibre.

With respect to the composite membrane 50, in some embodiments, for example, the membrane 30 has a thickness from 0.01 to 20 microns, such as from 0.5 to ten (10) microns, or such as from one (1) to five (5) microns, and the substrate material has a thickness from 30 to 200 microns, such as from 50 to 150 microns, or such as from 80 to 110 microns.

With respect to composite membranes, in some embodiments, for example, the membrane 30 is applied to the substrate 40. In some of these embodiments, for example, the application is by way of coating, casting, or laminating.

In some of embodiments, for example, the membrane layer is continuous. In some embodiments, for example, the membrane layer is discontinuous.

With respect to composite membranes 50, in some embodiments, for example, the membrane 30 extends into the pores of the substrate 40.

An exemplary method of manufacturing an embodiment of the membrane 30 includes casting a solution of polymeric material (such as one or more polysaccharides) as a film. In some embodiments, for example, the solution includes less than five (5) weight percent polymeric material, based on the total weight of solution. In some embodiments, for example, the solution includes less than two (2) weight percent polymeric material, based on the total weight of solution. In some embodiments, for example, the solution is an acidic aqueous solution. In some embodiments, the acid is an organic acid such as an organic acid having a total number of carbons of between one (1) and four (4). In some embodiments, for example, the acid includes acetic acid.

In some embodiments, for example, the resulting solution can be cast as a film on a flat plate to effect production of a membrane intermediate. Suitable casting surfaces include glass or Teflon™ or the like (e.g. a smooth substrate to which the polymer film will have a low adhesion). The solution is then dried to form a film. In other embodiments, for example, the resulting solution can be cast as a film on the substrate 40 to effect production of a membrane intermediate supported on the substrate 40 (which, upon conversion of the membrane intermediate to the membrane 30, results in the obtaining of the composite membrane 50).

In some embodiments, for example, the resulting solution can be coated onto the outer surface of a hollow fiber substrate to effect production of a membrane intermediate. Suitable hollow fibers include those fabricated from polysulphone, polyethersulphone, polyamide, polyimide, and polyetherimide. The solution is then dried to form a film on the hollow fiber surface. In other embodiments, for example, the resulting solution may be coated on the outer surface of the hollow fiber substrate to effect production of a membrane intermediate supported on the substrate (which, upon conversion of the membrane intermediate to the membrane, results in the obtaining of the composite membrane).

In those embodiments where the polymeric material includes polysaccharide material, in some of these embodiments, for example, the polymeric material includes chitosan. The following describes an exemplary method of manufacturing a membrane where the polymeric material of the polymeric phase is chitosan.

Chitosan is a generic term for deacetylation products of chitin obtained by treatment with concentrated alkalis. Chitin is the principal constituent of shells of crustaceans such as lobsters and crabs. In some embodiments, for example, chitosan is obtained by heating chitin, in the presence of an alkaline solution (such as, for example, an aqueous solution of sodium hydroxide) having an alkali concentration of 30 to 50% by weight, to a temperature of at least 60.degrees Celsius, with effect chitin is deacetylated. Chemically, chitosan is a linear polysaccharide composed of randomly distributed β-(1-4)-linked D-glucosamine (de-acetylated unit) and N-acetyl-D-glucosamine (acetylated unit). Chitosan readily dissolves in a dilute aqueous solution of an acid, such as acetic acid and hydrochloric acid, with the formation of a salt, but when contacted again with an aqueous alkaline solution, is again coagulated and precipitated. In some embodiments, for example, chitosan has a deacetylation degree of at least 50%, and in some of these embodiments, for example, chitosan has a deaccetylation degree of at least 75%.

Initially, chitosan is dissolved in a dilute aqueous acid solution. This effects protonation of the amino groups such that an ammonium salt is formed. Examples of suitable acids that can be utilized for protonation include inorganic acids such as hydrochloric acid, hydrobromic acid, sulfuric acid and phosphoric acid; and organic acids such as acetic acid, methanesulfonic acid, formic acid, propionic acid, oxalic acid, malonic acid, succinic acid, fumaric acid, maleic acid, glutaric acid, phthalic acid, isophthalic acid, terephthaic acid, trimesic acid, trimellitic acid, citric acid, aconitic acid, sulfobenzoic acid, pyromellitic acid and ethylenediaminetetraacetic acid.

The resulting solution is cast as a film onto a flat plate or onto a substrate material. The cast film can be contacted with an aqueous alkaline solution to neutralize the acidity and render the film less soluble or substantially insoluble in water, or can be air-dried and then contacted with the aqueous alkaline solution, with effect that the membrane intermediate is obtained. The neutralization with alkali effects deprotonation of chitosanium with effect that chitosan is restored.

In some embodiments, for example, the membrane intermediate has a dry thickness from 10 nanometres (0.01 microns) to 20 microns, such as from 0.5 to ten (10) microns, or such as from one (1) to five (5) microns. In some embodiments, for example, the substrate material has a thickness from 30 to 200 microns, such as from 50 to 150 microns, or such as from 80 to 110 microns.

The membrane intermediate is then contacted with a salt of a metal cation (such as silver ion or cuprous ion). In some embodiments, for example, the contacting includes immersing the membrane intermediate in an aqueous solution including a salt of a metal cation (such as one (1) to eight (8) M aqueous silver nitrate solution). The contacting effects disposition of metal cations into (for example, through chelation and/or complexing) and throughout the matrix of the membrane intermediate, and within its pores, if present, and effects formation of the gel, such that the membrane 30 is obtained.

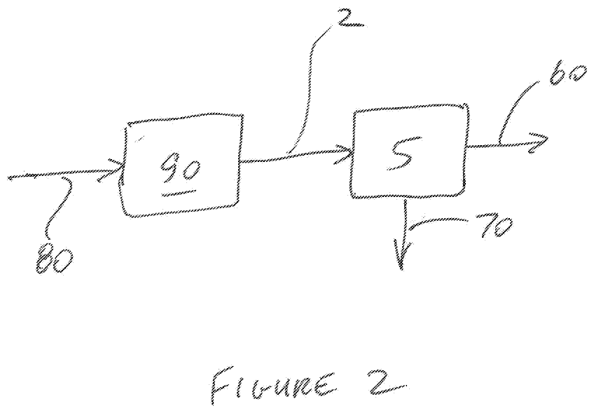

Referring to FIG. 2, in some embodiments, for example, the gaseous feed material 2 is a carrier agent-interacting material-depleted gaseous material that is obtained in response to treating of a carrier agent-interacting material-comprising material 80 within a system 90. In this respect, in some embodiments, for example, the process includes, within system 90, treating a carrier agent-interacting material-comprising material 80 that includes a carrier agent-interacting material to produce the carrier agent-interacting material-depleted gaseous material 2, and then, within system 5, fractionating the carrier agent-interacting material-depleted gaseous material 2 via the membrane 30. In some embodiments, for example, the carrier agent-interacting material-comprising material 80 is gaseous.

The carrier agent-interacting material is a material that, when disposed in mass transfer communication with (such as, for example, in proximity to) the membrane 30, is effective for interacting with the carrier agent of the membrane 30 (in some embodiments, for example, this interaction includes a reactive process whereby the carrier agent becomes converted to one or more other materials) with effect that performance, or service life, of the membrane 30 becomes compromised to an extent that continued use of the membrane 30 becomes commercially unsuitable. In some embodiments, for example, continued use of the membrane 30 becomes commercially unsuitable when there is a deterioration of membrane permeability, to the target permeating material, of at least 20% during a period of six (6) months. In this respect, in response to emplacement of the carrier agent-interacting agent in mass transfer communication with the carrier agent, the carrier agent-interacting agent is detrimental to the carrier agent.

In those embodiments where the carrier agent includes silver ion or cuprous ion, in some of these embodiments, for example, the carrier agent-interacting material includes at least one of gaseous diatomic hydrogen, gaseous acetylene, a gaseous alkene having a total number of carbon atoms of three (3) to six (6), inclusively, and a mixture of gaseous alkenes where each one of the gaseous alkenes, independently, has a total number of carbon atoms of three (3) to six (6), inclusively.

In those embodiments where the carrier agent includes silver ion or cuprous ion, in some of these embodiments, for example, the carrier agent-interacting material is gaseous diatomic hydrogen.

In those embodiments where the carrier agent includes silver ion or cuprous ion, in some of these embodiments, for example, the carrier agent-interacting material is gaseous acetylene.

In those embodiments where the carrier agent includes silver ion or cuprous ion, in some of these embodiments, for example, the carrier agent-interacting material is a gaseous alkene having a total number of carbon atoms of three (3) to six (6), inclusively.

In those embodiments where the carrier agent includes silver ion or cuprous ion, in some of these embodiments, for example, the carrier agent-interacting material is a mixture of gaseous alkenes where each one of the gaseous alkenes, independently, has a total number of carbon atoms of three (3) to six (6), inclusively.

In some embodiments, for example, the carrier agent-interacting material-depleted gaseous material 2 includes the carrier agent-interacting material, and the carrier agent-interacting material of the carrier agent-interacting material-depleted gaseous material 2, that is being supplied to the membrane 30, includes less than 20 ppm of carrier agent-interacting material, such as, for example, less than 5 ppm of carrier agent-interacting material, such as, for example, less than 1 ppm of carrier agent-interacting material.

Suitable treatment processes for treating the carrier agent-interacting material-comprising material 80 include absorption, adsorption, and chemical conversion.

In those embodiments where the carrier agent-interacting material includes gaseous diatomic hydrogen, in some of these embodiments, for example, suitable treatment processes for treating the carrier agent-interacting material-comprising material 80 include absorption, adsorption, and chemical conversion using hydrogen peroxide or other oxidizing agents.

In those embodiments where the carrier agent-interacting material includes gaseous acetylene, in some of these embodiments, for example, suitable treatment processes for treating the carrier agent-interacting material-comprising material 80 include catalytic conversion (acetylene is hydrogenated to ethane over a palladium-based catalyst), absorption with a dimethylformamide-water solution (see U.S. Pat. No. 3,004,629), and adsorption (e.g. on molecular sieves)

In some embodiments, for example, concentration of carrier agent-interacting material, within the carrier agent-interacting material-comprising material 80 being treated, is at least one (1) ppm, such as, for example, at least five (5) ppm, such as, for example, at least 20 ppm.

In some embodiments, for example, the treating of the carrier agent-interacting material-comprising material 80 is with effect that at least 70 weight percent (such as, for example, at least 85 weight percent, such as, for example, at least 95 weight percent) of the carrier agent-interacting material is removed.

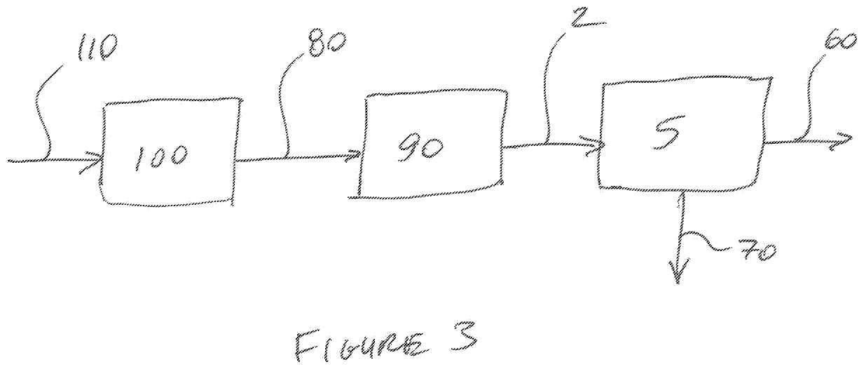

Referring to FIG. 3, in some embodiments, for example, the carrier agent-interacting material-comprising material is a product 80 of a methanol to olefin process (“MTO” process) implemented within a system 100, such that the carrier agent-interacting material-depleted gaseous material 2 is derived from the MTO process. A suitable MTO process is disclosed in U.S. Pat. No. 7,317,133. In this respect, in some embodiments, for example, the carrier agent-interacting material-comprising material is a product 80 obtained from conversion of methanol, such that the process includes converting, within system 100, methanol, of a methanol-comprising feed 110, to at least the carrier agent-interacting material-comprising material 80. The carrier agent-interacting material-comprising material 80 is supplied to, and treated within system 90 to produce the carrier agent-interacting material-depleted gaseous material 2. The carrier agent-interacting material-depleted gaseous material 2 is then supplied to system 5, with effect that the carrier agent-interacting material-depleted gaseous material 2 is fractionated via the membrane 30, as above-described, with effect that the permeate 60 is produced and the retentate 70 is produced.

In those embodiments where the carrier agent-interacting material-depleted gaseous material 2 is derived from the MTO process (as above-described), the carrier agent-interacting material-comprising material 80, as well as the carrier agent-interacting material-depleted gaseous material 2, includes an olefin-comprising material, and the olefin-comprising material includes at least one olefin. In this respect, the permeate 60 is defined by the olefinic material-enriched product, that is enriched in olefin-comprising material relative to the carrier agent-interacting material-depleted gaseous material 2, and the retentate 70 is defined by an olefinic material-depleted product that is depleted in olefin-comprising material relative to the carrier agent-interacting material-depleted gaseous material 2. In some embodiments, for example, each one of the at least one olefin, of the olefin-comprising material (of the carrier agent-interacting material-depleted gaseous material 2), independently, is an olefin having a total number of carbon atoms of from two (2) to eight (8). Suitable examples of an olefin having a total number of carbon atoms of from two (2) to eight (8) include ethylene, propylene, 1-butene, and 2-butene. In those embodiments where each one of the at least one olefin, of the olefin-comprising material (of the carrier agent-interacting material-depleted gaseous material 2), independently, is an olefin having a total number of carbon atoms of from two (2) to eight (8), in some of these embodiments, for example, each one of the at least one olefin, of the olefin-comprising material (of the carrier agent-interacting material-depleted gaseous material 2), independently, is an alpha olefin.

In those embodiments where the carrier agent-interacting material-depleted gaseous material 2 is derived from a MTO process, in some of these embodiments, for example, the first material is a first olefin and the second material is a second olefin, wherein:

the first olefin has a total number of “X” carbon atoms and the second olefin as a total number of “Y” carbon atoms;

each one of “X” and “Y”, independently, is a whole number that is equal to, or greater than, two (2); and

“X” is greater than “Y”.

In some of these embodiments, for example, the first olefin is propylene and the second olefin is ethylene.

In those embodiments where the carrier agent-interacting material-depleted gaseous material 2 is derived from a MTO process, in some of these embodiments, for example, the carrier agent-interacting material-comprising material 80, as well as the carrier agent-interacting material-depleted gaseous material 2, further includes a paraffin-comprising material, and the paraffin-comprising material includes at least one paraffin. In some of these embodiments, for example, the first material, of the carrier agent-interacting material-depleted gaseous material 2 (i.e. the gaseous feed material), is the olefin-comprising material and the second material, of the carrier agent-interacting material-depleted gaseous material 2 (i.e. the gaseous feed material), is the paraffin-comprising material. In this respect, the fractionation of the carrier agent-interacting material-depleted gaseous material 2 is with effect that the olefin-comprising material of the permeate 60, is enriched, relative to the olefin-comprising material of the carrier agent-interacting material-depleted gaseous material 2, and the paraffin-comprising material, of the retentate 70, is enriched, relative to the paraffin-comprising material of the carrier agent-interacting material-depleted gaseous material 2. In some embodiments, for example, the olefin-comprising material is an olefin having a total number of carbon atoms of from two (2) to eight (8), inclusively, and the paraffin-comprising material is a paraffin having a total number of carbon atoms of from one (1) to ten (10), inclusively. In some embodiments, for example, the olefin-comprising material is ethylene and the paraffin-comprising material is ethane. In some embodiments, for example, the olefin-comprising material is propylene and the paraffin-comprising material is propane.

Also in those embodiments where the carrier agent-interacting material-comprising gaseous material 2 is derived from the MTO process, in some of these embodiments, for example, the carrier agent-interacting material-comprising gaseous material 2 includes an olefin-comprising material, and the olefin-comprising material includes a first olefin and a second olefin, wherein:

the first olefin has a total number of “X” carbon atoms and the second olefin as a total number of “Y” carbon atoms;

each one of “X” and “Y”, independently, is a whole number that is equal to, or greater than, two (2);

“X” is greater than “Y”; and

the first material, of the carrier agent-interacting material-depleted gaseous material (i.e. the gaseous feed material), is the first olefin and the second material, of the carrier agent-interacting material-depleted gaseous material (i.e. the gaseous feed material), is the second olefin.

In some of these embodiments, for example, the first olefin is propylene and the second olefin is ethylene.

In some embodiments, for example, the permeate 60, the retentate 70, or each one of the permeate 60 and the retentate 70, independently, can be further treated in another membrane separation stage, for effecting further enrichment.

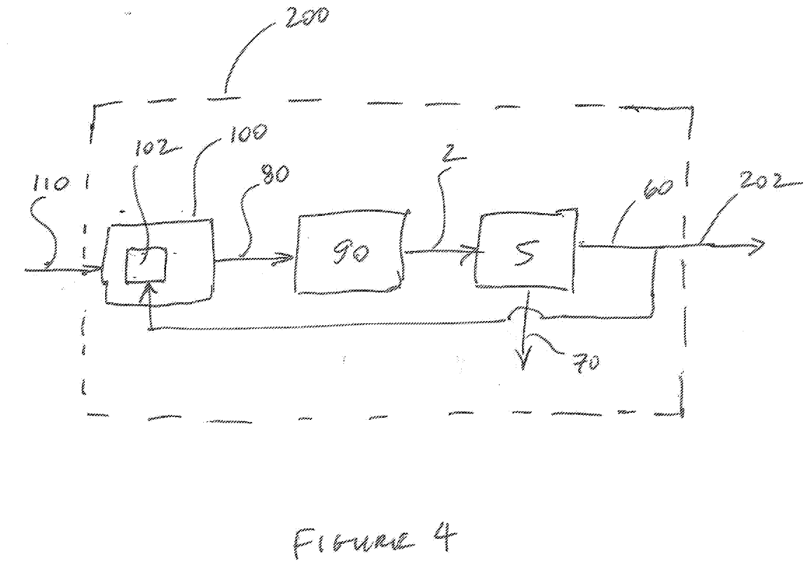

Referring to FIGS. 4 to 9, in some embodiments, for example, a process, for producing an olefin material rich product, is provided and implemented within system 200. The system 200 includes the system 100, the system 90, and the system 5.

The process includes converting, within system 100, methanol, of a methanol-comprising feed 110, to at least the carrier agent-interacting material-comprising material 80, wherein the carrier agent-interacting material-comprising material 80 includes an olefin-comprising material and a paraffin-comprising material. The carrier agent-interacting material-comprising material 80 is supplied to, and treated within system 90 to produce the carrier agent-interacting material-depleted gaseous material 2. The carrier agent-interacting material-depleted gaseous material 2 includes an olefin-comprising material and a paraffin-comprising material, and the olefin-comprising material and the paraffin-comprising material are derived from the carrier agent-interacting material-comprising material 80. The carrier agent-interacting material-depleted gaseous material 2 is then supplied to system 5, with effect that the carrier agent-interacting material-depleted gaseous material 2 is fractionated via the membrane 30, as above-described, with effect that the permeate 60 is produced and the retentate 70 is produced. The permeate 60 is defined by an olefinic material-enriched product, that is enriched in olefin-comprising material relative to the carrier agent-interacting material-depleted gaseous material. The retentate 70 is defined by an olefinic material-depleted product that is depleted in olefin-comprising material relative to the carrier agent-interacting material-depleted gaseous material.

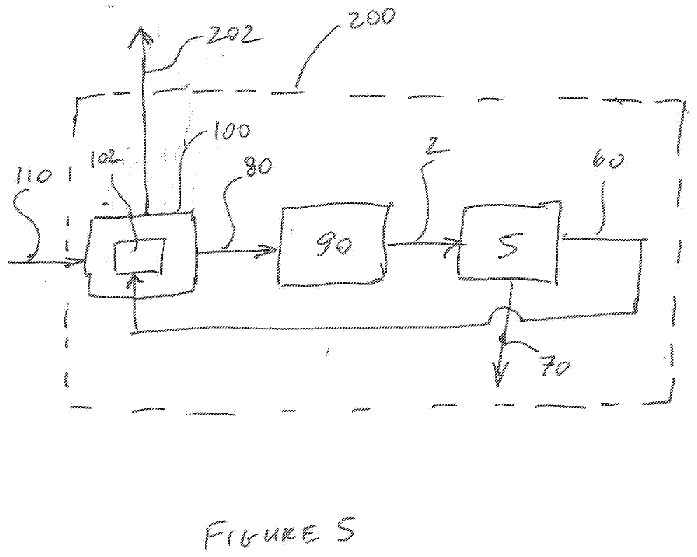

Referring to FIGS. 4 and 5, in some of these embodiments, for example, at least a portion of the permeate 60 is recycled to the MTO process. By recycling at least a portion of the permeate 60, a product (the olefinic material rich product) is obtainable that is further enriched in olefinic material than a product that is obtainable via a process that is implemented without the recycle. In some of these embodiments, for example, the MTO process includes a separation process (e.g. distillation) being implemented in a separation process-based unit operation 102 (e.g. distillation column) of the MTO system 100, and at least a portion of the permeate 60 is recycled to the separation process-based unit operation 102 (e.g. distillation column) of the MTO system 100. Referring to FIG. 4, in some of these embodiments, for example, the permeate 60 defines the olefinic material rich product. Referring to FIG. 5, in some of embodiments (such as in those embodiments where the entirety of the permeate 60 is recycled), for example, another product 202 of the system 200 (and, in some of these embodiments, more particularly, another product of the MTO system 100) defines the olefinic material rich product.

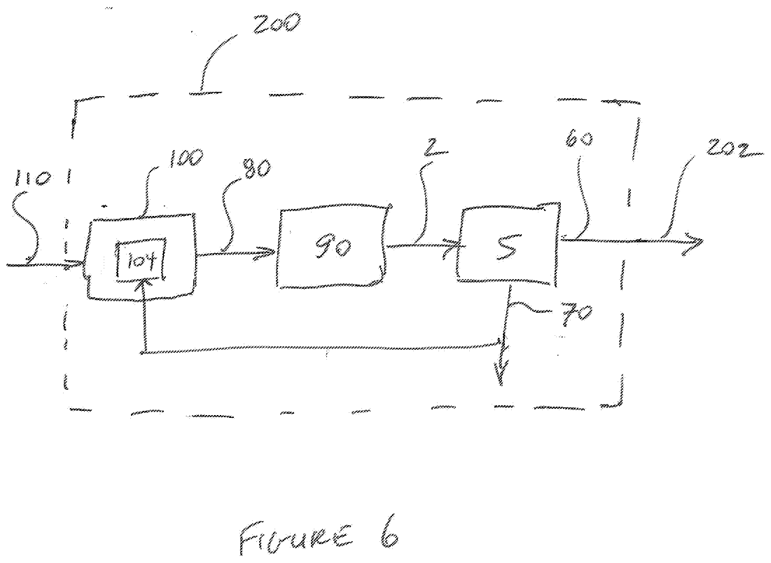

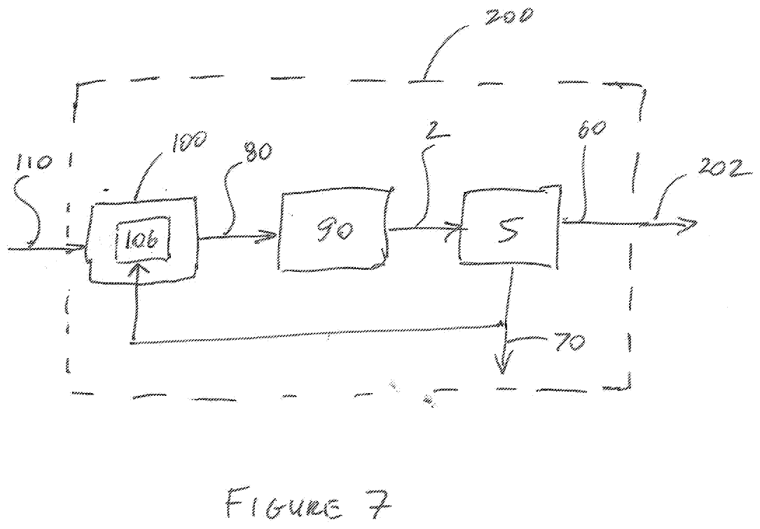

Referring to FIGS. 6 and 7, in some embodiments, for example, at least a portion of the retentate 70 is recycled to the MTO process for effecting recovery of olefinic material from the retentate 70, and thereby increasing recovery of olefinic material from the feed 110. Referring to FIG. 6, in some embodiments, for example, residual olefinic material in the retentate 70 can be recovered through a separation process (e.g. distillation) being implemented in a separation process-based unit operation 104 (e.g. distillation column) of the MTO system 100. In this respect, in some embodiments, for example, at least a portion of the retentate 70 is recycled to the separation process-based unit operation 104. Referring to FIG. 7, in some embodiments, for example, at least a portion of the paraffinic material in the retentate 70 can be converted to olefinic material, by recycling at least a portion of the retentate 70 to a chemical conversion-based unit operation 106 (e.g. a reactor, such as, for example, a cracker of the MTO system 100).

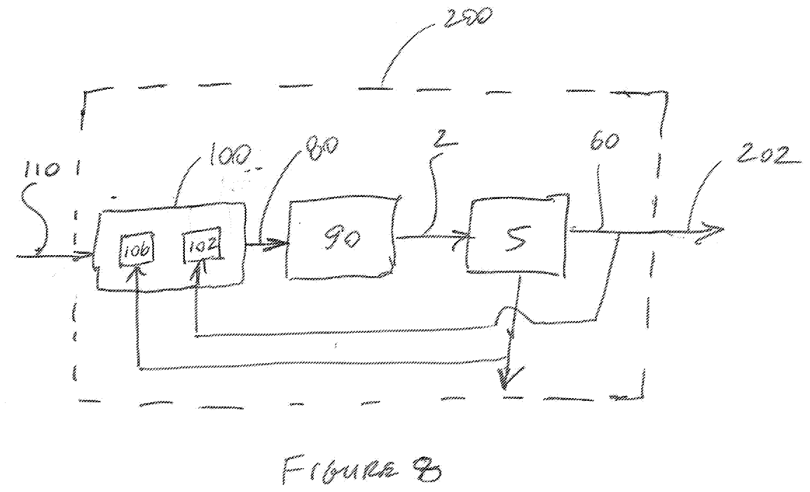

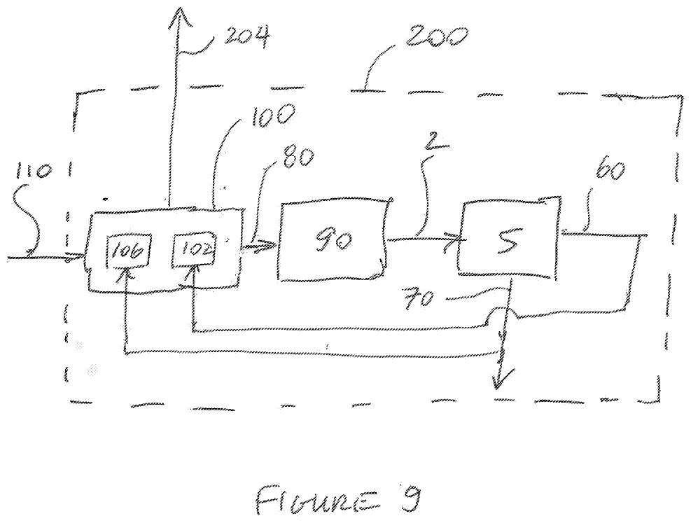

Referring to FIGS. 8 and 9, in some embodiments, for example, both of (i) at least a portion of the permeate 60 and (ii) at least a portion of the permeate are recycled to the MTO system 100. In this respect, in some embodiments, for example, at least a portion of the permeate 60 is recycled to the separation process-based unit operation 102 (e.g. distillation column) of the MTO system 100, and at least a portion of the retentate 70 is recycled to the chemical conversion-based unit operation 106 (e.g. a reactor, such as, for example a cracker) of the MTO system 100 with effect that at least a portion of the paraffinic material in the recycled retentate is converted to olefinic material, such that the olefin material rich product is produced. Referring to FIG. 8, in some of these embodiments, for example, the permeate 60 defines the olefinic material rich product. Referring to FIG. 9, in some of embodiments (such as in those embodiments where the entirety of the permeate 60 is recycled), for example, another product 204 of the system 200 (and, in some of these embodiments, more particularly, another product of the MTO system 100) includes the olefinic material rich product.

In the above description, for purposes of explanation, numerous details are set forth in order to provide a thorough understanding of the present disclosure. However, it will be apparent to one skilled in the art that these specific details are not required in order to practice the present disclosure. Although certain dimensions and materials are described for implementing the disclosed example embodiments, other suitable dimensions and/or materials may be used within the scope of this disclosure. All such modifications and variations, including all suitable current and future changes in technology, are believed to be within the sphere and scope of the present disclosure. All references mentioned are hereby incorporated by reference in their entirety.

1. A process for producing a target material-enriched product from a target material-comprising feed material, wherein the target material-comprising feed material includes a carrier agent-interacting material, comprising:
treating the target material-comprising feed material for effecting depletion of the carrier agent-interacting material within the target material-comprising feed material, with effect that a carrier agent-interacting material-depleted material is produced; and
fractionating the carrier agent-interacting material-depleted material via a membrane;
wherein:
the membrane includes a carrier agent to which the carrier agent-interacting material is detrimental in response to emplacement of the carrier agent-interacting material in mass transfer communication with the carrier agent.

2. The process as claimed in claim 1;
wherein:
the carrier agent-interacting material is reactive with the carrier agent.

3. The process as claimed in claim 1 or 2;
wherein:
the carrier agent includes a silver ion; and
the carrier agent-interacting material includes one or more alkenes; and
for each one of the one or more alkenes, the alkene has a total number of carbon atoms of three (3) to six (6), inclusively.

4. The process as claimed in claim 1;
wherein:
the carrier agent includes a cuprous ion; and
the carrier agent-interacting material includes one or more alkenes; and
for each one of the one or more alkenes, the alkene has a total number of carbon atoms of three (3) to six (6), inclusively.

5. The process as claimed in claim 1;
wherein:
the carrier agent-interacting material is gaseous.

6. The process as claimed in claim 1;
wherein:
the carrier agent includes a silver ion; and
the carrier agent-interacting agent is gaseous acetylene.

7. The process as claimed in claim 1;
wherein:
the carrier agent includes a silver ion; and
the carrier agent-interacting material is gaseous diatomic hydrogen.

8. The process as claimed in claim 1;
wherein:
the carrier agent includes a cuprous ion; and
the carrier agent-interacting material is gaseous acetylene.

9. The process as claimed in claim 1;
wherein:
the carrier agent includes a cuprous ion; and
the carrier agent-interacting material is gaseous diatomic hydrogen.

10. The process as claimed in claim 1;
wherein:
the target material-comprising feed material is a product of a MTO process.

11. The process as claimed in claim 1;
wherein:
the target material is olefin-comprising material.

12. The process as claimed in claim 1;
wherein:
the carrier agent-interacting material-depleted material is gaseous.

13. The process as claimed in claim 1;
wherein:
the concentration of carrier agent-interacting material, within the carrier agent-interacting material-comprising material, is at least one (1) ppm.

14. The process as claimed in claim 1;
wherein:
the treating of the carrier agent-interacting material-comprising material is with effect that at least 70 weight percent of the carrier agent-interacting material is removed.

15. A process for fractionating a gaseous feed material including an olefin-comprising material, the olefin-comprising material including a first olefin and a second olefin; and
fractionating the gaseous feed material via a membrane based on relative permeability as between the first and second olefins;
wherein:
the first olefin has a total number of “X” carbon atoms and the second olefin as a total number of “Y” carbon atoms;
each one of “X” and “Y”, independently, is a whole number that is equal to, or greater than, two (2);
“X” is greater than “Y”; and
the first olefin is characterized by a first permeability coefficient, and the second olefin is characterized by a second permeability coefficient, wherein the first permeability coefficient is greater than the second permeability coefficient.

16. The process as claimed in claim 15;
wherein:
the fractionation, is with effect that the separation factor for the separation of the first olefin from the second olefin, based on the first material, is at least two (2).

17. The process as claimed in claim 15;
wherein:
the membrane includes a carrier agent.

18. The process as claimed in claim 15;
wherein:
the first olefin is propylene; and
the second olefin is ethylene.

19. A process for recovering olefin-comprising material from a methanol-comprising material, comprising:
converting the methanol-comprising material to a gaseous material via a MTO process, wherein the gaseous material includes olefin-comprising material;
fractionating the gaseous material, via a membrane, with effect that: (i) a permeate is produced, the permeate being defined by an olefin material-enriched product that is enriched in olefin-comprising material relative to the gaseous material, and (ii) a retentate is produced, the retentate being defined by an olefin material-depleted product that is depleted in olefin-comprising material relative to the gaseous material; and
recycling at least a portion of the permeate to the MTO process.

20. The process as claimed in claim 19;
wherein:
the recycling includes recycling to a separation process-based unit operation of the MTO process.

21.-23. (canceled)
